(19) United States
(12) Patent Application Publication
(10) Pub. No.: US 2009/0118638 A1
(43) Pub. Date: May 7, 2009

(54) Device for Performing an Allergy Test

(76) Inventors: Gerhard Schindlbeck, Viernheim (DE); Claus Bachert, Kempen (DE)

Correspondence Address: KOLISCH HARTWELL, P.C., 200 PACIFIC BUILDING, 520 SW YAMHILL STREET, PORTLAND, OR, 97204, US

(21) Appl. No.: 11/885,086

(22) PCT Filed: Feb. 24, 2006
(86) PCT No.: PCT/EP06/01718

§ 371 Date: Aug. 24, 2007

(30) Foreign Application Priority Data
Feb. 24, 2005 (EP) 05004048.4

(51) Int. Cl. A61B 10/00

(52) U.S. Cl. 600/556

(57) ABSTRACT

The invention concerns a device (1) for performing an allergy test. Said device (1) comprises a container assembly (2) including several containers (3) designed to receive the allergens, and a mark transferable onto the skin which is used to associate specific allergens to specific allergy sites on the skin of a living being undergoing an allergy test. The invention aims at improving said device (1) so that the allergy test sites on the skin can be constantly marked very legibly, and so that the corresponding marks can be readily eliminated from the skin immediately after the allergy test. Therefor, the transferable mark is formed by at least one marking unit (6) which is removably fixed on said device (1), and whereof the side, facing towards the skin during the allergy test, is at least partly coated with an adhesive substance, such that the device (1) can be spaced apart while the marking unit (6) remains on the skin.

### I. TECHNICAL FIELD

The present invention relates to a device for performing an allergy test in which the skin of a living being undergoing the test, in particular of a test person, is brought into contact with allergens, and the skin is scratched or damaged before, after or during the application of the allergens thereto, in order to cause a reaction of the test person to the allergens. In addition, the present invention relates also to a device for performing an allergy test in which the skin does not have to be scratched or damaged.

### II. TECHNICAL BACKGROUND

A device for performing an allergy test of the type in question is already known from DE 43 28 112 C1. The known device has a cover unit that can be placed onto the skin, and a container unit which is connected to the cover unit in an articulated manner and which comprises several containers for receiving the allergens. After the cover unit has been applied, for example to the forearm of a test person, the skin is scratched or damaged with the aid of a scratching or pricking tool via the apertures in the cover unit. A closing sheet that closes off the containers of the container unit can then be pulled off, and the container unit can be folded onto the cover unit. In this way, the allergens located in the containers pass through the apertures and onto the damaged areas of the skin, and it is then possible to wait for the reactions of the test person to the various allergens.

If, after application of the allergens to the skin and after scratching of the latter, the known device is to be removed before any allergic reactions can be observed, the device has a marking that can be transferred to the skin in order to allocate specific allergens to specific allergy test sites. This transferable marking is either a transferable dye that adheres well to the skin, or a film that can be transferred like a transfer print. Transferable markings of this kind have the disadvantage that, on the one hand, they may be illegible when transferred or may wipe off and, on the other hand, they often remain on the skin for longer than is necessary, which many test persons dislike for esthetic reasons.

In connection with the device according to DE 43 28 112 C1, it is also known to fix the container unit to the cover unit after the allergy test sites on the skin have been scratched. This fixing is done either by partial adhesion or by means of a Velcro-type strip, which is wound round the test person's arm, for example. Fixing of this kind is relatively complicated for the person performing the allergy test, on the one hand because aids such as adhesives or Velcro-type strips have to be kept ready and, on the other hand, because several maneuvers have to be performed. This complexity adversely affects the costs of the allergy test and also increases the time needed to perform it.

### III. DISCLOSURE OF THE INVENTION

### a) Technical Object

It is therefore an object of the present invention to create a device with which an allergy test is performed and which, on the one hand, ensures that the allergy test sites on the skin are at all times marked in an easily legible manner and, on the other hand, allows the corresponding markings to be easily removed from the skin immediately after completion of the allergy test. A further object of the present invention is to create a device with which an allergy test is performed and which allows a container unit to be fixed on a cover unit in the simplest possible way. An additional object of the present invention is to create a device with which an allergy test is performed and which as a whole simplifies the test in terms of the handling of the device.

### b) Solution to the Object

This object is achieved by a device having the features of claims 1, 16 and 24. Further embodiments of the present invention are set forth in the dependent claims.

According to the invention, a device for performing an allergy test is proposed, comprising a container unit with several containers for receiving allergens, in which device a transferable marking is formed by at least one marking unit which is secured releasably to the device and whose side facing toward the skin during the allergy test is at least partially provided with an adhesive, such that the device can be removed from the skin of a living being undergoing the test while at the same time the at least one marking unit remains adhering to the skin. The separation of the marking unit from the rest of the device takes place during the removal of the device from the skin.

In a manner known per se, the container unit of the device according to the invention is preferably flat and is also preferably made of a thin, flexible material, for example plastic, that can be adapted to the contour of the allergy test site, for example the forearm of a test person. The marking unit can be made from the same material as the container unit and can be secured to a side edge of the container unit, for example along a perforated tear line. This affords the advantage that container unit and marking unit can be produced in one and the same production step. After the container unit has been removed from the allergy test site, the at least one marking unit remains like an adhesive strip on the skin of the test person, such that the information applied on the side of the marking unit directed away from the skin can be used to clearly allocate a specific allergen to a specific allergy test site for the purpose of evaluating the allergy test. Accordingly, the allocation information is no longer applied directly to the skin by means of a dye or in the manner of a transfer print. The marking unit designed in the manner of an adhesive strip simply adheres to the skin of the living being undergoing the test and can easily be pulled off from the skin when the allergy test evaluation has been completed.

A marking unit within the meaning of the present invention can also be used in devices which, in addition to the container unit, comprise a cover unit with several apertures for demarcating the allergy test sites. In these devices, the cover unit can be placed in a manner known per se onto the skin of the living being undergoing the test, and the container unit for its part can be placed onto the cover unit in such a way that the apertures are substantially in alignment with the containers. In devices of this kind, the marking unit can either be secured releasably to a side edge of the container unit or to a side edge of the cover unit. Since container unit and cover unit preferably have the same geometry in a plan view, the marking unit protrudes past the sides of the container unit or of the cover unit, depending on whether it is secured to the cover unit or to the container unit. In cases where the marking unit is arranged on the container unit, the particular advantage achieved is that, since the marking unit is held down like an adhesive tape, the device according to the invention lies with continuously planar contact on what is in most cases a curved surface of the skin at the allergy test site.

In its position when protruding from the container unit or from the cover unit, the marking unit according to the invention lies preferably in the plane included by the container unit or the cover unit. To minimize the size of the device according to the invention, and therefore the space it takes up during transport and storage, it is conceivable for the marking unit to be arranged initially in a state folded onto the container unit or the cover unit. When the device is finally to be used for performing an allergy test, the marking unit is pivoted or folded, through substantially 180° about the side edge on which it is secured, into its position in which it protrudes from the container unit or the cover unit.

Particularly where several substantially parallel rows of containers or of containers and apertures are provided in a manner known per se, there is preferably a marking unit arranged on each of two opposite side edges of the container unit or, if appropriate, of the cover unit. The allocation information of the respective marking unit then relates to that row of containers or apertures or allergy test sites lying closest to the marking unit.

It is also conceivable not only for the marking unit to be arranged releasably on the container unit or the cover unit, but also for the containers to be releasable from the container unit. In this variant, the container unit can be placed or affixed onto the skin, and the skin is then scratched with the aid of a pricking tool through the containers. The container unit is pulled off from the skin in such a way that the individual containers of the container unit, and also the marking unit, remain adhering to the skin. After a certain period of action, the individual containers can finally be removed, and the allergy test can be evaluated with the aid of the allocation information on the marking unit.

The device according to the invention can also have a frame part with a frame opening which, when the device is placed onto the skin, defines the work area in which the allergy test sites are located. The container unit, the cover unit and the at least one marking unit are in this case each secured to a respective outer side edge of the frame part. In particular, in order to minimize the size of the device, and therefore the space it takes up during transport and storage, the container unit and the cover unit can be secured to the frame part in an articulated manner, such that they can be folded or pivoted through substantially 180° into the frame opening.

In the same way as in the variant without a frame part, the marking unit in the variant comprising a frame part can also be secured releasably along a perforated tear line, but not to the container unit or to the cover unit, and instead to the frame part. The container unit and the cover unit are preferably secured to opposite sides of the frame part. The marking unit can be pivoted through substantially 180° between a position in which it protrudes from the frame part to a position in which it lies substantially within the frame opening. Especially in cases where there are substantially parallel rows of containers and apertures, the marking unit is preferably located on two opposite side edges of the frame part.

The adhesive applied to the side of the marking unit directed toward the skin can be an adhesive film, the latter being covered by a removable protective sheet that protects it from contaminants. The entire side of the marking unit directed toward the skin is preferably coated with the adhesive film. However, it is also conceivable for the coating to be provided only in some parts, as long as it ensures a secure hold on the skin of the living being undergoing a test.

According to the invention, a device for performing an allergy test is also proposed that comprises a container unit with several containers for receiving allergens, and a cover unit with several apertures for demarcating allergy test sites, and in which the side of the cover unit onto which the container unit can be placed is at least partially provided with an adhesive film, the latter being covered by a peel-off sheet that has through-holes substantially in alignment with the apertures in the cover unit. The advantage of such a device is that in a single maneuver, namely removal of the peel-off sheet, it is possible to ensure that the container unit, when placed onto the cover unit, is fixed securely on the latter and cannot slip out of place. Running or smudging of allergens between cover unit and container unit is avoided. In addition, after a sufficient period of action, the container unit and cover unit can be removed from the allergy test site and disposed of as a coherent, compact unit.

The cross sections of the through-holes in the peel-off sheet preferably lie completely within the cross sections of the apertures in the cover unit. Moreover, the through-holes and the apertures can have the same cross-sectional shape and the same cross-sectional midpoint. If, in addition to this, the cross-sectional surface areas of the through-holes are smaller than the cross-sectional surface areas of the apertures, the advantage achieved by this is that the skin, when scratched with a pricking tool via the through-holes and the apertures, is always scratched in an area of the skin lying centrally with respect to the through-holes in the cover unit. This makes overlapping of allergic reactions at adjacent allergy test sites less likely.

The cross sections of the through-holes and/or of the apertures can have, for example, a circular shape, a rectangular shape or a square shape. The through-holes and apertures do not necessarily need to have identical cross-sectional shapes. Depending on requirements, combinations of different cross-sectional geometries are also conceivable. It is advantageous for the through-holes in the peel-off sheet to have a cross shape. This permits crosswise scratching of the allergy test sites on the skin, such that the skin is reliably scratched at least at the midpoint of the cross.

In a particularly advantageous manner, the allergy test sites can be sealed off or isolated from one another if each container of the container unit has a sealing lip in its edge area. When the container unit is placed onto the cover unit, the sealing lips preferably engage through the respective aperture, such that the sealing lips can each be brought into contact with the skin around the respective allergy test site. In this way, the allergy test sites are effectively sealed off from one another, such that an allergen allocated to one test site cannot end up at another allergy test site. In a particularly advantageous manner, the sealing lip according to the invention can be generated when a closing sheet that closes off the containers is bonded by heat-sealing to the container unit.

According to the invention, a device for performing an allergy test is additionally proposed in which the containers of a container unit are closed off by a preferably sheet-like material both on their side directed toward the skin during the allergy test and also on their side directed away from the skin during an allergy test, said sheet-like material being able to be pierced by means of a pricking tool such that, by piercing a container of the container unit lying on the skin, a pricking needle of the pricking tool can be wetted with allergens and, moreover, the skin can be damaged, thereby allowing the allergen to be introduced into the skin.

A device of this kind can advantageously be handled without placing the container unit onto a cover unit and without a separate step for opening the containers. A marking unit of the kind explained above can be provided if required. Alternatively, it is possible, in a manner known per se, for the side of the container unit directed toward the skin during the allergy test to be provided with allocation information that is transferable in the manner of a transfer print for the purpose of allocating specific allergens to specific allergy test sites.

In a device of this kind, it is particularly advantageous if the containers, on their side directed away from the skin during the allergy test, each have a chimney-shaped or pipe-shaped continuation that guides the pricking tool as it pierces the container. Here, the inner cross-sectional shape of the continuation preferably corresponds substantially to the cross-sectional shape of the area of the pricking tool that is to be guided. To permit free mobility of the pricking tool the latter is guided with a certain play.

### c) Illustrative Embodiments

Several embodiments of the present invention are described below by way of example and with reference to the attached drawings, in which:

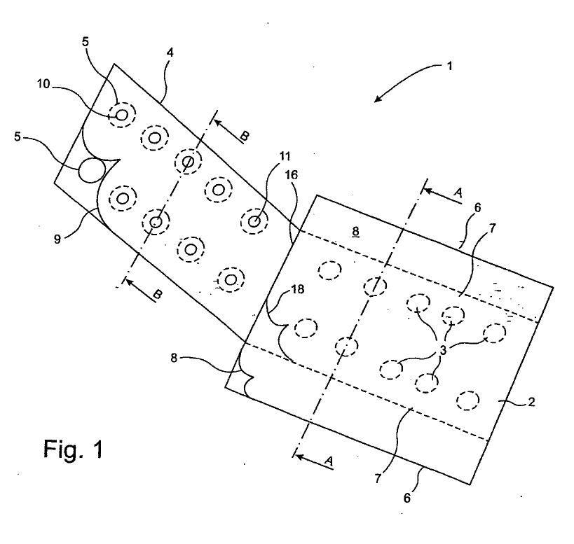

FIG. 1 shows a perspective view of a first embodiment of the device according to the invention;

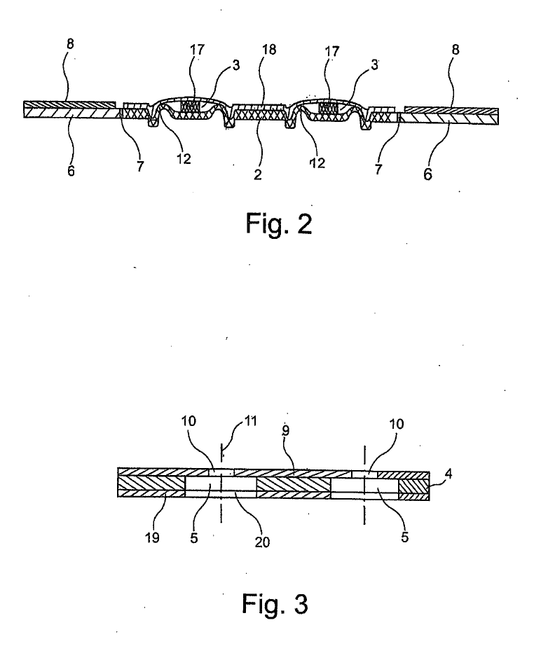

FIG. 2 shows a cross-sectional view along section A-A in FIG. 1;

FIG. 3 shows a cross-sectional view along section B-B in FIG. 1;

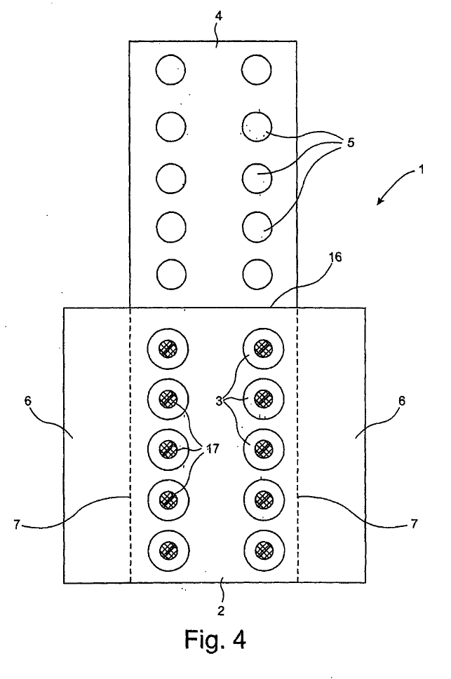

FIG. 4 shows a plan view of the front face of the device according to FIG. 1 without a closing sheet and peel-off sheet;

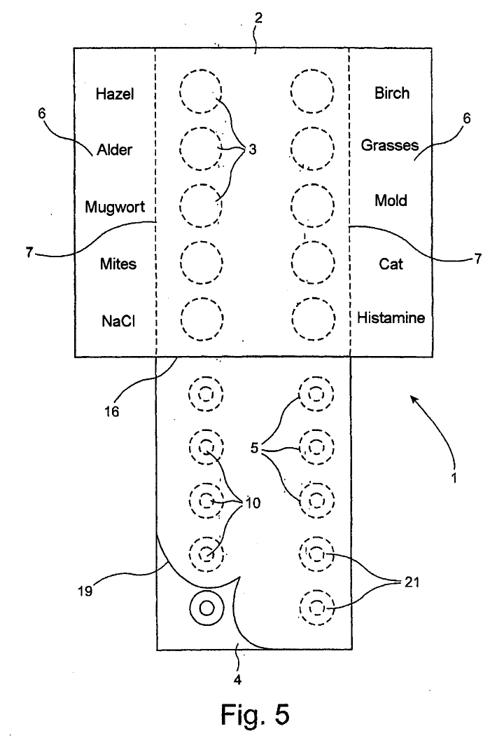

FIG. 5 shows a plan view of the rear face of the device according to FIG. 1;

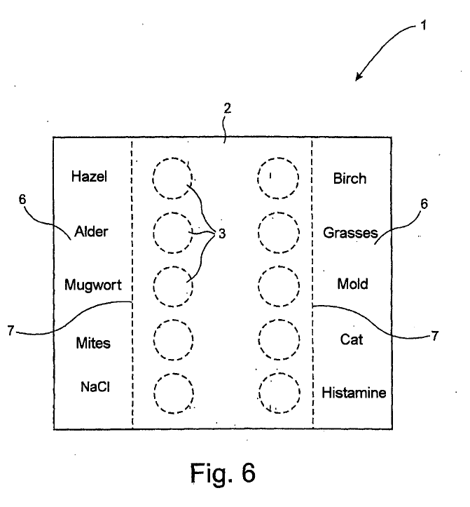

FIG. 6 shows a plan view of the side, directed away from the skin, of a second embodiment of the device according to the invention; and

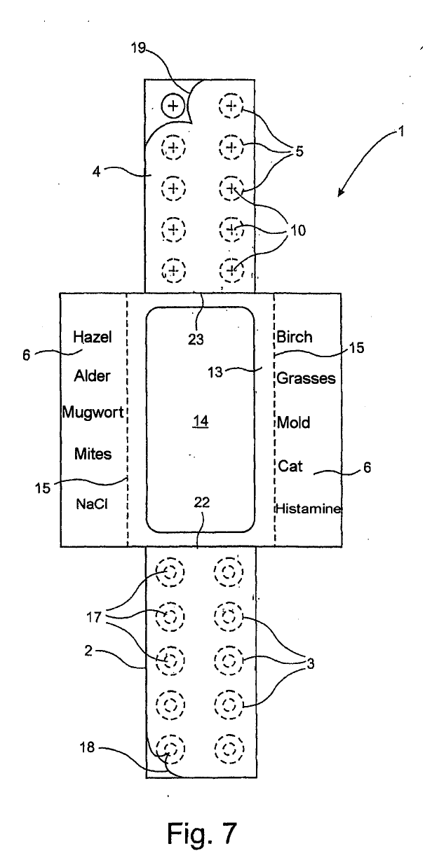

FIG. 7 shows a plan view of a third embodiment of the device according to the invention.

Identical parts that have identical functions are indicated by the same reference numbers in the drawings.

A first embodiment of the device 1 according to the invention is shown in FIG. 1. The device 1 is composed of a flat, rectangular container unit 2 functioning as chamber part, a flat, rectangular cover unit 4 functioning as template part, and two flat, rectangular marking units 6 arranged like wings on the mutually opposite long sides of the cover unit 4. The container unit 2, the cover unit 4 and the marking units 6 are flexible and able to bend, such that they can be adapted to curvatures or bulges of the surface of the skin. In the embodiment shown, the aforementioned units are made of a thin plastic which, if appropriate, is coated in a suitable way.

In a manner known per se, the container unit 2 is connected to the cover unit 4 so as to be able to be folded or turned with the aid of a hinge 6. When the container unit 2 is folded onto the cover unit 4, the two marking units 6 thus protrude past the long sides of the cover unit 4 and can be brought into contact with the skin. The marking units 6 are each secured releasably to the container unit 2 along a perforated tear line 7. Alternatively, the releasability can be provided by thinning of the material between marking units 6 and container unit 2. In addition, the tear lines 7 can function as hinges, such that the marking units 6 can be folded through ca. 180° from the position shown in FIGS. 1, 4 and 5 onto the container unit 2. It is conceivable for them to be folded onto the front face visible in FIG. 1 or onto the rear face not visible in FIG. 1. Preferably, the marking units 6 are folded inward onto the front face shown in FIG. 1, since in this case the marking units 6 can be stowed between cover unit 4 and container unit 2 when said container unit 2 is folded onto the cover unit 4. Irrespective of the direction in which the marking units 6 are folded, their folding inward has a space-saving effect, which means that the device 1 according to the invention does not require more space than do the devices known from the prior art for performing allergy tests.

The container unit 2 comprises, in a manner known per se, containers 3 for receiving allergens. As can be seen from FIGS. 1, 4 and 5, there are two parallel rows in total, each with five containers 3. However, any other desired geometries of container arrangements are also conceivable, as long as they meet the requirements of performing the allergy test. Examples of these are container arrangements whose geometries, seen in a plan view, display the contours of animals or comic figures or a heart shape. Container arrangements of this kind may help allay any fears children might have about the allergy test. In addition, the containers 3 formed in the container unit 2 can be provided in any desired shapes, colors and numbers.

FIG. 2 shows the cross-sectional view, along the section A-A in FIG. 1, through two containers 3. In the embodiment shown, a spongy tissue 17 soaked with a specific allergen is located in each of the containers 3. Instead of being in liquid form, however, the allergens can also be arranged in the containers in solid, gel-like or pasty form, without the spongy tissue 17. In addition, it is alternatively conceivable for the containers 3 to hold a pricking needle on which an allergen is located. It is then possible to damage the skin by pressing the respective container 3 from the lower side of the container unit 2 in FIG. 2. In the embodiment shown, the containers 3 are closed off by means of a closing sheet 18 preferably made of aluminum. The closing sheet 18 ensures that the allergens contained in the spongy tissues 17 are preserved and can be used for as long a period as possible for the purposes of an allergy test. To open the containers 3, the sheet can be pulled off by hand from the container unit 2. For this purpose, the closing sheet 18 can have a surface area that is slightly larger than that of the container unit 2. The part of the closing sheet 18 protruding past the side edges of the container unit 2 can then be used as a tab for pulling off the protective sheet 18.

The two marking units 6 can also be seen in FIG. 2, where they are connected to the container unit 2 via the perforated tear line 7. On the top face of each of the marking units 6 in FIG. 2 there is a peel-off protective sheet 8, which covers an adhesive film located on the top face of each of the marking units 6 in FIG. 2. This adhesive film allows the marking units 6 to be affixed to the skin of a living being undergoing a test, in particular to the forearm of a test person.

On the lower face of the marking units 6 in FIG. 2, i.e. on the rear face not visible in FIGS. 1 and 4, and shown in FIG. 5, there are, according to the invention, items of allocation information showing what type of allergen is contained in a specific container 3. As will be seen from FIG. 5, the allocation information is for this purpose applied to the marking units 6 in such a way that it is located directly adjacent to the respective container 3 whose allergen it is intended to identify. In the illustrative embodiment shown, the allocation information is in each case applied as a word that represents the allergy for which the test person, is testing the allergen located in the respective container 3. As will be seen from FIG. 5, these words are “Hazel”, “Alder”, “Mugwort”, “Mites”, “NaCl”, “Birch”, “Grasses”, “Mold”, “Cat” and “Histamine”. It is of course also possible, within the context of the present invention, for the allocation information to be provided by other allergen indicators in text and/or symbol format.

FIG. 3 shows the cross section B-B, from FIG. 1, through the cover unit 4. The cover unit 4 has apertures 5 which, when the container unit 2 is folded onto the cover unit 4, are substantially in alignment with the containers 3 in a manner known per se. On the top face of the cover unit 4 in FIG. 3, which is also seen in FIG. 1, there is a peel-off sheet 9 that covers an adhesive film located on the top face of the cover unit 4 in FIG. 3. This adhesive film can be applied across substantially the entire surface, or only part of the surface, of the cover unit 4. For example, it is conceivable for only a few spots of adhesive film to be provided. The adhesive film covered by the peel-off sheet 9 ensures that the container unit 2 folded onto the cover unit 4 is fixed securely on the cover unit 4.

In addition, the peel-off sheet 9 has through-holes 10 that are each in alignment with the apertures 5 in the cover unit 4 and, as viewed from above in FIG. 3, lie with their cross-sectional shape completely within the cross-sectional shape of the respective aperture 5. The particular advantage of this configuration is described further below in connection with the function of the device 1. In the embodiment shown, the apertures 5 and the through-holes 10 have a circular geometry, and the respective midpoints 11 of the circles are coincident. Other cross-sectional geometries are also conceivable here, for example square, rectangular, elliptic or cross-shaped forms. It is also conceivable for the apertures 5 and also the through-holes 10 to have cross-sectional geometries differing from one another.

On the lower face of the cover unit 4 in FIG. 3, there is a further peel-off sheet 19, which covers another adhesive film located on the lower face of the cover unit 4 in FIG. 3. This adhesive film, applied at least in parts, allows the cover unit 4 to be safely secured or affixed to the surface of the test person's skin. The peel-off sheet 19 for its part has through-openings 20 which, in the embodiment shown, correspond to the apertures 5 in terms of number, position, cross-sectional geometry and size. It is also conceivable to design the through-openings 20 with other cross-sectional geometries and sizes or to design the peel-off sheet 19 entirely without through-openings 20, since the peel-off sheet 19 is removed completely from the cover unit 4 before the actual allergy test is performed, in particular before scratching the skin.

FIG. 4 shows a plan view of the front face of the device 1 according to the invention as seen in FIG. 1, with the closure sheet 18 having been removed from the container unit 3, the protective sheets 8 having been removed from the marking units 6, and the peel-off sheet 9 having been removed from the cover unit 4. Accordingly, FIG. 4 shows the open containers 3 with the spongy tissues 17 located therein. Moreover, the apertures 5 in the cover unit 4 can be seen, their outer edge areas no longer covered by the peel-off sheet 9.

FIG. 5 shows a plan view of the rear face of the device 1 according to the invention not visible in FIGS. 1 and 4. As in FIG. 4, the container unit 2 and the cover unit 4 again lie in one plane. In FIG. 5, the containers 3 arranged in the container unit 2 are visible only from the direction of their closed rear face. The allocation information “Hazel”, “Alder”, “Mugwort”, “Mites”, “NaCl”, “Birch”, “Grasses”, “Mold”, “Cat” and “Histamine” is visible on the two marking units 6. Circular areas 21 of the peel-off sheet 9 are visible through the apertures 5 provided in the cover unit 4.

The way in which the device 1 according to FIGS. 1-5 functions and is used in the context of performing an allergy test will now be described by way of example:

After a surface of a test person's skin has been prepared, for example on the forearm, by cleaning thereof, the peel-off sheet 19 is removed from the cover unit 4, and the latter is pressed onto the skin surface and secured thereon with the aid of the exposed adhesive film. With the aid of a pricking or scratching tool known per se, the surface of the skin is then scratched by way of the through-holes 10 located in the peel-off sheet 9. For this purpose, the pricking tool can be moved, for example, in a straight line and without interruption along the rows of through-holes 10 shown in FIG. 1. After the surface of the skin has been scratched, the peel-off sheet 9 is removed in order to expose the adhesive film lying below it. Moreover, the containers 3 are opened by peeling off the closing sheet 18, and the adhesive films on the marking units 6 are exposed by removing the protective sheets 8. Thereafter, the container unit 2 and marking units 6 can be folded through ca. 180° around the hinge 16 in such a way that the containers 3 are substantially in alignment with the apertures 5, and the allergens in the spongy tissues 17 arrive on the allergy test sites that are demarcated on the surface of the skin by the apertures 5.

On account of the adhesive film on the face of the cover unit 4 directed away from the skin, the container unit adheres securely to the cover unit 4 and cannot slip out of place. Container unit 2 and cover unit 4 form a compact, manageable unit which is no longer connected only linearly via the hinge 16, but also in a planar manner. In addition, the two marking units 6 are pressed firmly onto the forearm of the test person, such that they adhere to the surface of the skin.

After a certain duration of action on the allergy test sites, the compact and manageable unit made up of container unit 2 and cover unit 4 can be removed from the surface of the skin by being gripped and removed while at the same time pressing or holding the marking units 6 firmly on the surface of the skin. The container unit 2 is torn off from the marking units 6 along the perforated tear line 7, which marking units 6 remain on the surface of the test person's skin for evaluation of the allergy test. The reactions of the test person to the individual allergens can then be read off by the person conducting the test and can be documented with the aid of the allocation information located on the marking units 6. After the documentation of the test result has been completed, the marking units 6 can be withdrawn from the surface of the skin, such that no markings (in some cases completely removable only after quite a long time) remain on the forearm of the test person. If so required, the marking units 6 can even be used for documentation purposes by gluing them or otherwise placing them in a medical file.

When the skin is scratched with the pricking tool, the through-holes 10 present in the peel-off sheet 9 afford a particular advantage. The skin is scratched more or less centrally with respect to the larger apertures 5. In this way, it is possible to avoid scratching the skin at the edge of the apertures 5, which means that it is not possible for two scratched allergy test sites to come too close to one another. The reliability of the scratching of the skin can also be increased in a particularly advantageous way. It is conceivable to use cross-shaped through-holes 10 instead of circular through-holes 10. The skin can then be scratched by the pricking tool being guided through the cross-shaped through-hole 10 in two straight scratch lines extending substantially perpendicular to one another. The surface of the skin is reliably scratched at least at the point of intersection of the two straight scratch lines.

To reliably ensure that the allergen located between skin surface and cover unit 4 and allocated to a specific allergy test site does not reach another allergy test site, which would of course falsify the result of the allergy test, the container unit 2 has a sealing lip 12, which can be seen in FIG. 2. The sealing lip 12 lies in the edge area of each container 3 and preferably also forms the side wall thereof. In plan view, it can either have the same geometry as the apertures 5 in the cover unit 4 or, if so required, can be designed with a geometry differing from this. As can be seen in FIG. 2, the sealing lip 12 at the top of FIG. 2 protrudes above the surface of the container unit 2, such that, when the container unit 2 and cover unit 4 are placed together, it engages through the apertures 5 provided in the cover unit 4 and can come into contact with the surface of the skin. The respective allergy test site is in this way safely isolated from other allergy test sites by means of the contact between the sealing lip 12 and the surface of the skin. In the embodiment shown, the sealing lip 12 is particularly advantageously produced at the same time as the closing sheet 18 is heat-sealed onto the container unit 2. A heat-sealing die, in this case with a circular die face, is directed from above in FIG. 2 onto the closing sheet 18, pressing the latter onto the container unit 2. This results in displacement of material, particularly on the container unit 2, which leads to the formation of the sealing lip 12 shown in FIG. 2.

Within the context of the present invention, it is alternatively possible for the marking units 6 to be arranged not on the container unit 2, but instead on the long sides of the cover unit 4. This does not appreciably alter the above-described function of the device 1. Before the cover unit 4 is placed onto the surface of the skin, the protective sheets 8 in addition to the peel-off sheet 19 have to be pulled off from the marking units 6.

FIG. 6 shows a second embodiment of a device 1 according to the invention, the direction of the view corresponding to that of FIG. 5, i.e. the rear face of the device 1 is shown. In this embodiment, the cover unit 4 present in the first embodiment has been omitted without being replaced, but it is otherwise unchanged from the first embodiment. According to the invention, the device 1 allows an allergy test to be performed without the skin being scratched or pricked, which many test persons dislike. For this purpose, the spongy tissue 17 located in the containers 3 contains not only the respective allergen, but also a substance that opens the skin barrier chemically and/or biologically and/or physically. An example of such a substance is dimethyl sulfoxide. In this case the scratching or pricking of the skin is replaced by the skin barrier being opened during the period of action after the container unit 2 has been placed onto the surface of the skin. The marking units 6 are used, in the same way as has been described in connection with the first embodiment, for allocating specific allergens to specific allergy test sites.

In the embodiment according to FIG. 6, however, it is also possible for the allergy test to be performed by scratching or pricking of the skin. For this purpose, a substantially circular spongy tissue soaked with allergen, or a gel-like allergen, can be arranged in each of the containers 3 in such a way that, by placing the container unit 2 onto the skin, it can be transferred to the latter, such that the spongy tissue or the gel-like allergen can be pierced by means of a pricking tool after the container unit 2 has been removed, thus applying the allergen onto or into the skin. A cover unit functioning as a template part is not required, because the pattern of the arrangement of containers 3 in the container unit 2 is as it were transferred to the skin.

In the context of the present invention, a possible modification of the embodiment according to FIG. 6 is for the rear face shown in FIG. 6 and the front face not shown in FIG. 6 to be provided in each case with a sheet which can be pierced by means of a pricking needle and which in each case closes off the individual containers 3. To apply the allergens onto or into the skin, it is then not necessary to pull off one of the sheets. Instead, after the device 1 has been applied to the skin, the containers 3 are simply pierced by means of the pricking needle in such a way that, in a single step, the pricking needle is wetted with allergen and, moreover, the skin is scratched by means of the wetted pricking needle. If so required, the thus modified embodiment can also be formed without the two marking units 6. Alternatively, the front face of the device 1 not visible in FIG. 6 can then be provided, for example, with allocation information that can be transferred to the skin in the manner of a transfer print, in order to allocate specific allergens to specific allergen test sites.

FIG. 7 shows a third embodiment of a device 1 according to the invention. In contrast to the first embodiment, the third embodiment has a flat and substantially rectangular frame part 13 that encloses a frame opening 14. Marking units 6 according to the invention are arranged on the two opposite long sides of the frame part 13 via a perforated tear line 15. A control unit 2, whose structure corresponds to that described in connection with the first embodiment, is secured by means of a hinge 22 to the lower short side of the frame part 13 in FIG. 7. A cover unit 4, which corresponds to that described in connection with the first embodiment, is secured by means of a hinge 23 to the upper short side of the frame part 13 in FIG. 7.

The individual steps involved in performing an allergy test by means of the embodiment according to FIG. 7 are described below by way of example:

The protective sheets 8 are first removed from the sides (not shown in FIG. 7) of the two marking units 6. With the aid of the adhesive films thus exposed, the device 1 is secured to the surface of the skin, for example a forearm, of a test person. The peel-off sheet 19 is then removed from the cover unit 4, and the latter is folded through substantially 180° about the hinge 23 into the frame opening 14. The adhesive film of the cover unit 4 previously covered by the peel-off sheet 19 ensures that said cover unit 4 is held securely on the surface of the skin. It is alternatively conceivable here for the peel-off sheet 19, including the adhesive film of the cover unit 4 that it covers, to be omitted and not replaced, since the affixed marking units 6 already provide a certain hold for the device 1 on the surface of the skin. After the cover unit 4 has been placed onto the skin, the individual allergy test sites are scratched or pricked in the same way as has been described in connection with the first embodiment. In FIG. 7, the cross-shaped through-holes 10 in the peel-off sheet 9 can be seen through the apertures 5. As has been described above, this arrangement reliably ensures that the skin is scratched at the point of intersection of the cross.

After the skin has been scratched, the peel-off sheet 9 (not visible in FIG. 7) is peeled off so as to completely expose the apertures 5. Moreover, in order to open the containers 3, the closing sheet 18 is removed from the container unit 2. Thereafter, the container unit 2 with the open containers 3 can be folded through substantially 180° about the hinge 22 into the frame opening 14 and thus onto the cover unit 4. The adhesive film of the cover unit 4 previously covered by the peel-off sheet 9 ensures that the container unit 2 is held securely on the cover unit 4. A compact and manageable unit is obtained comprising the frame part 13, the cover unit 4 and the container unit 2. After a certain period of action, this compact and manageable unit can be completely removed from the skin by means of the marking units 6 being pressed down or held down and the compact manageable unit being torn off along the two perforated tear lines 15.

### LIST OF REFERENCE NUMBERS

1 device
2 container unit
3 container
4 cover unit
5 aperture
6 marking unit
7 tear line
8 protective sheet
9 peel-off sheet
10 through-holes
11 midpoint of circle
12 sealing lip
13 frame part
14 frame opening
15 tear line
16 hinge
17 spongy tissue
18 closing sheet
19 peel-off sheet
20 through-openings
21 area of the peel-off sheet 9
22, 23 hinge

1. A device for performing an allergy test, the device comprising:
a container unit (2) with several containers (3) for receiving allergens, and
a marking that is transferable onto the skin for the purpose of allocating specific allergens to specific allergy test sites on the skin of a living being undergoing a test, characterized in that the transferable marking is formed by at least one marking unit (6) which is secured releasably to the device and whose side facing toward the skin during the allergy test is at least partially provided with an adhesive, such that the device can be removed while the marking unit (6) remains on the skin.

2. The device of claim 1, further comprising a cover unit (4) with several apertures (5) for demarcating the allergy test sites, the cover unit (4) configured to be placed onto the skin of the living being undergoing a test, and the container unit (2) configured to be placed onto the cover unit (4) in such a way that the apertures (5) are substantially in alignment with the containers (3).

3. The device of claim 1, characterized in that the marking unit (6) is secured releasably to a side edge of the container unit (2), such that the container unit (2) can be removed while the marking unit (6) remains on the skin.

4. The device of claim 2, characterized in that the marking unit (6) is secured releasably to a side edge of the container unit (2) or of the cover unit (4), such that the container unit (2) and the cover unit (4) can be removed while the marking unit (6) remains on the skin.

5. The device of claim 3, characterized in that the at least one marking unit (6) is secured to the container unit (2) along a perforated tear line (7).

6. The device of claim 4, characterized in that the at least one marking unit (6) is secured to the container unit (2) or the cover unit (4) along a perforated tear line (7).

7. The device of claim 3, characterized in that the at least one marking unit (6) is pivotable through a range of substantially 180° between a first position, in which it lies on the container unit (2), and a second position, in which it protrudes from the side edge of the container unit (2).

8. The device of claim 4, characterized in that the at least one marking unit (6) is pivotable through a range of substantially 180° between a first position, in which it lies on the container unit (2) or the cover unit (4), and a second position, in which it protrudes from the side edge of the container unit (2) or of the cover unit (4).

9. The device of claim 3, characterized in that there is a marking unit (6) arranged on each of two opposite side edges of the container unit (2).

10. The device of claim 4, characterized in that there is a marking unit (6) arranged on each of two opposite side edges of the container unit (2) or of the cover unit (4).

11. The device of claim 3, characterized in that the containers (3) are releasable from the container unit (2), such that the container unit (2) can be removed while the containers (3) remain on the skin.

12. The device of claim 4, characterized in that the containers (3) are releasable from the container unit (2), such that the container unit (2) and the cover unit (4) can be removed while the containers (3) remain on the skin.

13. The device of claim 2, further comprising a frame part (13) with a frame opening (14), wherein the container unit (2), the cover unit (4) and the at least one marking unit (6) are each secured on an outer side edge of the frame part (13).

14. The device of claim 13, characterized in that the container unit (2) and the cover unit (4) are secured to the frame part (13) in an articulated manner, such that the container unit and the cover unit are foldable through a range of substantially 180° into the frame opening (14).

15. The device of claim 13, characterized in that the at least one marking unit (6) is secured to the frame part (13) along a perforated tear line (15).

16. The device of claim 13, characterized in that the container unit (2) and the cover unit (4) are secured to opposite sides of the frame part (13).

17. The device of claim 13, characterized in that the at least one marking unit (6) is pivotable through a range of substantially 180° between a first position, in which it lies substantially in the frame opening (14), and a second position, in which it protrudes from the side edge of the frame part (13).

18. The device of claim 13, characterized in that there is a marking unit (6) arranged on each of two opposite side edges of the frame part (13).

19. The device of claim 1, characterized in that the adhesive is an adhesive film covered by a removable protective sheet (8).

20. The device of claim 1, further comprising a substantially circular spongy tissue soaked with allergen, or a gel-like allergen, arranged in each of the containers (3) in such a way that, by placing the container unit (2) onto the skin, it can be transferred to the latter, such that the spongy tissue or the gel-like allergen can be pierced by means of a pricking tool after removal of the container unit (2) to introduce the allergen into the skin.

21. A device for performing an allergy test, comprising a container unit (2) with several containers (3) for receiving allergens, and a cover unit (4) with several apertures (5) for demarcating allergy test sites, the cover unit (4) configured to be placed onto the skin of a living being undergoing a test, and the container unit (2) configured to be placed onto the cover unit (4) in such a way that the apertures (5) are substantially in alignment with the containers (3), characterized in that the side of the cover unit (4) onto which the container unit (2) can be placed is at least partially provided with an adhesive film, covered by a peel-off sheet (9) that has through-holes (10) substantially in alignment with the apertures (5).

22. The device of claim 21, characterized in that the cross sections of the through-holes (10) lie completely within the cross sections of the apertures (5).

23. The device of claim 21, characterized in that the through-holes (10) and the apertures (5) have the same cross-sectional shape and the same cross-sectional midpoint (11).

24. The device of claim 21, characterized in that the cross sections of one or more of the through-holes (10) and the apertures (5) have a circular shape.

25. The device of claim 21, characterized in that the cross sections of one or more of the through-holes (10) and the apertures (5) have a rectangular or square shape.

26. The device of claim 21, characterized in that the cross sections of one or more of the through-holes (10) and the apertures (5) have a cross shape.

27. The device of claim 21, characterized in that each container (3) has a sealing lip (12) in its edge area.

28. The device of claim 27, characterized in that, when the container unit (2) is placed onto the cover unit (4), each sealing lip (12) engages through the respective aperture (5) such that it can be brought into contact with the skin around the respective allergy test site to provide a seal in relation to adjacent allergy test sites.
